(19) United States
(12) Patent Application Publication
(10) Pub. No.: US 2019/0314288 A1
(43) Pub. Date: Oct. 17, 2019

(54) POROUS POLYMER MICROSPHERES FOR PREVENTING OR TREATING SOFT TISSUE DISEASES AND METHOD FOR MANUFACTURING THE SAME

(71) Applicants: KOREA UNIVERSITY RESEARCH AND BUSINESS FOUNDATION, Seoul (KR); ORTHO HEAL CO., LTD., Seoul (KR)

(72) Inventors: Hae-Ryong SONG, Seoul (KR); Jae Young LEE, Seoul (KR); Sungeun KIM, Seoul (KR); Hak Jun KIM, Seoul (KR); Young-Pil YUN, Seoul (KR)

(73) Assignees: KOREA UNIVERSITY RESEARCH AND BUSINESS FOUNDATION, Seoul (KR); ORTHO HEAL CO., LTD., Seoul (KR)

(21) Appl. No.: 15/736,084

(22) PCT Filed: May 4, 2017
(86) PCT No.: PCT/KR2017/004723

§ 371 Date: Dec. 13, 2017

(30) Foreign Application Priority Data
Aug. 12, 2016 (KR) 10-2016-0103183

(51) Int. Cl. A61K 9/50; A61K 9/00; A61K 31/167; A61K 31/192; A61K 31/12; A61P 29/00; A61K 9/10

(52) U.S. Cl. CPC A61K 9/5031; A61K 9/0019; A61K 31/167; A61K 9/10; A61K 31/192; A61K 31/12; A61P 29/00; A61K 9/5026

(57) ABSTRACT

Disclosed are porous polymer microspheres for preventing or treating soft tissue diseases including a biodegradable polymer scaffold having a three-dimensional network structure in which pores having a size of 5 to 100 μm are connected to one another, and a drug for treating soft tissue diseases incorporated in the network structure of the biodegradable polymer scaffold.

### TECHNICAL FIELD

The present invention relates to porous polymer microspheres for preventing or treating soft tissue diseases and a method for manufacturing the same.

### BACKGROUND ART

Soft tissues mean soft sites or tissues thereof that surround bones or joints, and encompass membranes, tendons, chords, ligaments and cartilage that surround bones. Among them, cartilage includes a cartilage cell and a cartilage substrate. The cartilage is present in the form of a cartilage joint between bones involving little movement, but in sites requiring much movement, a synovial joint is present, along with a synovial fluid between cartilage surfaces. In addition, the cartilage serves as a buffer against a given force due to high elasticity and helps movement of joints under the condition of almost no friction due to considerably low coefficient of friction of joint cartilage.

Damage of soft tissues is caused by exterior shock or strong distortion, pulling, pressure, excessive exercise, prolonged fatigue, aging or wounds. Damaged soft tissues can be treated by surgical operation or using mechanical devices, but cannot be completely treated and cause serious discomfort in routine life such as gait disturbance. In order to overcome these surgical limitations, surgical methods using autologous cells or implantation of natural cartilage have been developed. However, these methods are restricted due to drawbacks such as isolation of limited numbers of cells, pathological conditions of isolated sites, limited proliferation of isolated cells, as well as cell mutation during implantation, and are not easy to utilize due to high costs.

Methods for soft tissue regeneration include oral administration of drugs and direct injection of drugs into diseased sites. However, oral administration of drugs cannot obtain sufficient regeneration effects because the amount of drugs delivered to diseased sites requiring regeneration is extremely small, and direct injection of drugs into diseased sites has a drawback of side-effects because a great amount of drug is injected at a time.

In an attempt to solve these problems, tissue engineering approaches using biodegradable porous polymer scaffolds arose as an alternative. It is known that biodegradable porous polymer scaffolds mimic natural extracellular matrices and are considerably useful for recovering and regenerating tissues such as blood vessels, tendons, cartilage, chords, ligaments and bones. These scaffolds have a benefit of high cell-adherence, but drawbacks of bad mechanical properties and slow cartilage regeneration rates. Therefore, there is a need for development of novel therapeutic drugs that can exhibit rapid regeneration and avoid side effects.

### PRIOR ART DOCUMENT

### Patent Document

Korean Patent No. 10-1105285

Korean Patent Publication Laid-open No. 2014-0147880

### DISCLOSURE

### Technical Problem

Therefore, the present invention has been made in view of the above problems, and it is one object of the present invention to provide porous polymer microspheres including a drug for preventing or treating soft tissue diseases.

It is another object of the present invention to provide a method for manufacturing the porous polymer microspheres.

### Technical Solution

In accordance with the present invention, the above and other objects can be accomplished by the provision of porous polymer microspheres for preventing or treating soft tissue diseases including a biodegradable polymer scaffold having a three-dimensional network structure in which pores having a size of 5 to 100 μm are connected to one another, and a drug for treating soft tissue diseases incorporated in the network structure of the biodegradable polymer scaffold.

According to the present invention, the porous polymer microspheres may include the drug in an amount of 2 to 30 parts by weight with respect to 10 parts by weight of the biocompatible polymer.

According to the present invention, the porous polymer microspheres may have a particle size of 200 to 1,000 μm.

According to the present invention, the porous polymer microspheres may have a porosity of 10 to 90%.

According to the present invention, the porous polymer microspheres may have a porosity of 60 to 90%, a pore size of 10 to 40 μm and a particle size of 200 to 500 μm.

According to the present invention, the drug for treating soft tissue diseases may be selected from transforming growth factor (TGF), platelet derived growth factor (PDGF), basic fibroblast growth factor (bFGF), vascular endothelial growth factor (VEGF), insulin like growth factor (IGF), bone morphogenetic protein-7 (BMP-7), anti-inflammatory peptide, aspirin, mefenamic acid, diclofenac sodium, indomethacin, naproxen, acetaminophen, ketoprofen, loxoprofen, piroxicam, ibuprofen, simvastatin, atorvastatin, fluvastatin, lovastatin, copper-peptide, prostaglandin, tramadol, celecoxib, glucosamine, chondroitin, diacerein, methotrexate, cyclosporine, an Janus Kinase 3 inhibitor (JAK-3 inhibitor), rituximab, tocilizumab, salazosulfapyridine (SASP), bucillamine, leflunomide, infliximab, etanercept, adalimumab, prednisolone, antiflammin 2, curcumin, sulfasalazine, lactoferrin, kartogenin, ibuprofen or a mixture thereof.

According to the present invention, the biocompatible polymer may be selected from the group consisting of: any one selected from polyglycolic acid (PGA), polylactic acid (PLA), poly(lactide-co-glycolide) (PLGA), polycaprolactone (PCL), polyamino acid, polylactide, polyphosphazine, polyiminocarbonate, polyphosphoester, polyanhydride, polyorthoester, polyhydroxyvalerate, polyhydroxybutyrate, hyaluronic acid, cellulose, heparin, collagen, alginate and chitosan; polymers blended with two or more thereof; and copolymers of two or more thereof.

According to the present invention, the soft tissue disease may be selected from Achilles tendinitis, rotator cuff tendinitis, patellar tendinitis, sprains, ligamentitis, degenerative osteoarthritis and inflammatory arthritis.

According to the present invention, the porous polymer microspheres may release the drug for treating soft tissue diseases when injected in vivo.

In another aspect of the present invention, provided is a method for manufacturing porous polymer microspheres for preventing or treating soft tissue diseases including 1) dissolving a biocompatible polymer and a drug for treating soft tissue diseases in an organic solvent to prepare a polymer solution, 2) homogenizing the polymer solution and an aqueous gelatin solution to prepare an emulsion, 3) pouring the emulsion as a discontinuous phase and a polyvinyl alcohol solution as a continuous phase into a fluidic device to produce polymer microspheres including the biocompatible polymer, the drug and gelatin, and 4) stirring the polymer microspheres in 38-50° C. water to release gelatin from the polymer microspheres.

### Effects of the Invention

The porous polymer microspheres according to the present invention enable drugs effective for regeneration of damaged soft tissues to be directly injected into diseased sites in need of treatment. In addition, since the drug is slowly released, a desired amount of drug can be delivered to the diseased site for a prolonged time, excellent regeneration effects can be directly expressed in soft tissues, and side-effects can be prevented because they are made of a biodegradable material. In addition, the porous polymer microspheres according to the present invention can provide personalized medical treatment because the rate and amount of released drug can be regulated by controlling porosity or the concentration of contained drug depending on conditions of diseased sites.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a scanning electron microscope image of porous polymer microspheres of Example 1;

FIG. 2 is a scanning electron microscope image of porous polymer microspheres of Example 2;

FIG. 3 is a scanning electron microscope image of porous polymer microspheres of Example 3;

FIG. 4 shows scanning electron microscopy results to identify variations in pore size of porous polymer microspheres depending on concentration of a drug;

FIG. 5 is a confocal laser scanning microscope image of porous polymer microspheres of Example 3;

FIG. 6 shows drug release behavior test results of porous polymer microspheres (KGN/PMS) of Example 1;

FIG. 7 shows drug release behavior test results of porous polymer microspheres (IBU/PMS) of Example 2;

FIG. 8 shows drug release behavior test results of porous polymer microspheres (Cur/PMS) of Example 3;

FIG. 9 shows measurement results of expression amounts of MMP3, MMP13, COX-2 and ADAMTS-5 in vitro, in order to identify inflammation suppression effects of porous polymer microspheres (KGN/PMS) of Example 1;

FIG. 10 shows measurement results of expression amounts of MMP3, MMP13, COX-2, ADAMTS-5, IL-6 and TNF-α in vitro, in order to identify inflammation suppression effects of porous polymer microspheres (KGN/PMS) of Example 2;

FIG. 11 shows measurement results of expression amounts of MMP3, MMP13, COX-2 and ADAMTS-5 in vitro, in order to identify inflammation suppression effects of porous polymer microspheres (KGN/PMS) of Example 3;

FIG. 12 shows measurement results of expression amounts of aggrecan, COL1A1, COL2A1 and COL10A1 in vitro, in order to identify cartilage differentiation expression of porous polymer microspheres (KGN/PMS) of Example 1;

FIG. 13 shows results of X-ray imaging to identify cartilage production resulting from treatment of porous polymer microspheres (KGN/PMS) of Example 2;

FIG. 14 shows results of micro-CT imaging to identify cartilage production resulting from treatment of porous polymer microspheres (KGN/PMS) of Example 2;

FIG. 15 shows results of imaging regarding the excised knee in order to identify cartilage production resulting from treatment of porous polymer microspheres (KGN/PMS) of Example 2;

FIG. 16 shows results of hematoxylin & eosin (H&E) staining regarding a knee fragment in order to identify cartilage production resulting from treatment of porous polymer microspheres (KGN/PMS) of Example 2;

FIG. 17 shows results of safranin O staining regarding a knee fragment in order to identify cartilage production resulting from treatment of porous polymer microspheres (KGN/PMS) of Example 2; and

FIG. 18 shows measurement results of expression amounts of MMP3, MMP13, COX-2, ADAMTS-5, IL-6 and TNF-α in rat blood in order to identify inflammation suppression effect of porous polymer microspheres (KGN/PMS) of Example 2.

### BEST MODE

Hereinafter, the present invention will be described in more detail.

The present invention provide porous polymer microspheres for preventing or treating soft tissue diseases that includes a biodegradable polymer scaffold having a three-dimensional network structure in which pores having a size of 5 to 100 μm are connected to one another, and a drug for treating soft tissue diseases incorporated in the network structure of the biodegradable polymer scaffold.

When the porous polymer microspheres according to the present invention are injected in vivo, the porous polymer microspheres may release the drug for treating soft tissue diseases. Drug release is carried out through pores of the porous polymer microspheres, or is carried out when the biodegradable polymer scaffold (hereinafter referred to as “polymer scaffold”) is degraded in vivo.

The release conditions, such as amount or rate, of the drug can be controlled depending on the type of polymer constituting the polymer scaffold, the density of network structure, porosity and pore size. Accordingly, the porosity and pore size of porous polymer microspheres, and concentration of incorporated drug can be suitably controlled depending on severity of diseased sites in need of treatment and then applied to patients.

The porous polymer microspheres according to the present invention may contain the drug in an amount of 2 to 30 parts by weight, with respect to 10 parts by weight of the biocompatible polymer. Disadvantageously, when the content of the drug is less than the range, the amount of drug is excessively low, and when the content of the drug exceeds the range, the amount of drug released within a short time is excessively high.

The porous polymer microspheres according to the present invention have a porosity of 10 to 90% and a particle size of 200 to 1000 μm. When the particle size is less than the range, the drug cannot be continuously released in vivo due to excessively less amount of drug that can be contained therein, and there is thus an inconvenience that the drug should be frequently administered, and when the particle size exceeds the range, it is not easy to manufacture microspheres.

The porous polymer microspheres according to the present invention preferably have a porosity of 60 to 90%, a pore size of 10 to 40 μm, and a particle size of 200 to 500 μm. The porous polymer microspheres satisfying the ranges enable the drug to be easily released in vivo.

The porous polymer microspheres according to the present invention may have a pore size of 5 to 100 μm. When the pore size is less than the range, the drug is not easily released due to excessive small pore size, and when the pore size exceeds the range, the drug is disadvantageously released at a time. The pore size is particularly preferably 10 to 80 μm.

The drug for treating soft tissue diseases according to the present invention may be selected from transforming growth factor (TGF), platelet derived growth factor (PDGF), basic fibroblast growth factor (bFGF), vascular endothelial growth factor (VEGF), insulin like growth factor (IGF), bone morphogenetic protein-7 (BMP-7), anti-inflammatory peptide, aspirin, mefenamic acid, diclofenac sodium, indomethacin, naproxen, acetaminophen, ketoprofen, loxoprofen, piroxicam, ibuprofen, simvastatin, atorvastatin, fluvastatin, lovastatin, copper-peptide, prostaglandin, tramadol, celecoxib, glucosamine, chondroitin, diacerein, methotrexate, cyclosporine, an Janus Kinase 3 inhibitor (JAK-3 inhibitor), rituximab, tocilizumab, salazosulfapyridine (SASP), bucillamine, leflunomide, infliximab, etanercept, adalimumab, prednisolone, antiflammin 2, curcumin, sulfasalazine, lactoferrin, kartogenin, ibuprofen or a mixture thereof.

Specifically, the transforming growth factor (TGF) may be TGF-alpha, TGF-beta, TGF-beta 1, TGF-beta 2 or TGF-beta 3, the platelet derived growth factor (PDGF) may be PDGFA, PDGFB, PDGFC or PDGFD, the basic fibroblast growth factor (bFGF) may be FGF2 or FGF-beta, the vascular endothelial growth factor (VEGF) may be VEGF-A, VEGF-B, VEGF-C or VEGF-D, and the insulin like growth factor (IGF) may be IGF-1 or IGF-2.

The biocompatible polymer of the porous polymer microspheres according to the present invention may be selected from the group consisting of: any one selected from poly(glycolic acid) (PGA), polylactic acid (PLA), poly (lactide-co-glycolide) (PLGA), polycaprolactone (PCL), polyamino acid, polylactide, polyphosphazine, polyiminocarbonate, polyphosphoester, polyanhydride, polyorthoester, polyhydroxyvalerate, polyhydroxybutyrate, hyaluronic acid, cellulose, heparin, collagen, alginate and chitosan; polymers blended with two or more thereof; and copolymers of two or more thereof.

The drug for treating soft tissue diseases according to the present invention is particularly preferably kartogenin, ibuprofen, curcumin or a mixture thereof.

The soft tissue diseases according to the present invention may be selected from Achilles tendinitis, rotator cuff tendinitis, patellar tendinitis, sprains, ligamentitis, degenerative osteoarthritis and inflammatory arthritis, but the present invention is not limited thereto.

Regarding the present invention, the term “preventing or treating soft tissue diseases” may mean facilitating regeneration of soft tissues such as cartilage, or preventing inflammation thereof.

Meanwhile, the porous polymer microspheres for preventing or treating soft tissue diseases according to the present invention can be manufactured by a method that includes: 1) dissolving a biocompatible polymer and a drug for treating soft tissue diseases in an organic solvent to prepare a polymer solution; 2) homogenizing the polymer solution and an aqueous gelatin solution to prepare an emulsion; 3) pouring the emulsion as a discontinuous phase and a polyvinyl alcohol solution as a continuous phase into a fluidic device to produce polymer microspheres including the biocompatible polymer, the drug and gelatin; and 4) stirring the polymer microspheres in 38-50° C. water to release gelatin from the polymer microspheres.

First, 2 to 30 parts by weight of the drug is mixed with 10 parts by weight of the biocompatible polymer and the mixture is dissolved in the organic solvent to prepare the polymer solution.

The organic solvent may include at least one selected from dimethylsulfoxide, dichloromethane, tetrahydrofuran and N,N-dimethylformamide, but the present invention is not limited thereto.

Next, the aqueous gelatin solution and the polymer solution are homogenized to prepare the emulsion.

The aqueous gelatin solution may be a 1 to 10 wt % aqueous gelatin solution. When the content of gelatin is less than the range, it is not easy to grow pores of microspheres due to excessively high content of gelatin, and when the content exceeds the range, it is not easy to produce microspheres.

According to the present invention, an emulsion stabilizer may be further added during homogenization. The emulsion stabilizer may be a commonly used emulsion stabilizer, preferably polyvinyl alcohol.

According to the present invention, in 2) homogenization, 3 to 15 parts by weight of gelatin may be added with respect to 10 parts by weight of the biocompatible polymer. When the content of gelatin is less than the range with respect to the biocompatible polymer, it is not easy to grow pores and to obtain the desired porosity. When the content of gelatin exceeds the range, it is not easy to contain the drug due to excessively large pores.

According to the present invention, 0.5 to 3 parts by weight of the emulsion stabilizer may be added with respect to 10 parts by weight of the biocompatible polymer. When the content of emulsion stabilizer is not within this range, it is not easy to form microspheres having the desired size in the present invention.

In addition, by controlling the content of gelatin and/or the drug with respect to the content of the biocompatible polymer, the amount and rate of released drug can be suitably regulated.

In addition, the homogenization is carried out using a homogenizer at 8,000 to 20,000 rpm for 30 seconds to 10 minutes, but the present invention is not limited thereto.

According to the present invention, the biocompatible polymer and the drug for treating soft tissue diseases have been described above.

Next, the emulsion as a discontinuous phase and the polyvinyl alcohol solution as a continuous phase are poured into the fluidic device to produce the target polymer microspheres. When the produced microspheres are stirred in 38-50° C. water, the gelatin is released from the microspheres and pores are created in areas where gelatin has been present, thus leading to formation of porous polymer microspheres.

At this time, the flow rates of the discontinuous and continuous phases may be each 0.2 to 1 mL/min, preferably 0.5 mL/min. A flow rate within this range enables microspheres having excellent porosity, and desired size and porosity to be formed.

The microspheres produced by the method have a porosity of 10 to 90%, a pore size of 5 to 100 μm, and a particle size of 200 to 1,000 μm, preferably a porosity of 60 to 90%, and the microspheres may have a pore size of 10 to 40 μm and a particle size of 100 to 400 μm.

Soft tissue diseases can be prevented or treated by administering an effective amount of the porous polymer microspheres according to the present invention.

The term “effective amount” as used herein means an amount of an effective (active) ingredient or a pharmaceutical composition that induces biological or medical reactions in tissue systems, animals or humans considered by researchers, veterinarians, doctors or other clinicians, and includes an amount that induces symptoms of the corresponding diseases or disorders. The effective amount and administration number of the effective ingredient according to the present invention can be changed according to desired effects. Thus, the optimal dosage can be easily determined by those skilled in the art and can be regulated by various factors such as type and severity of diseases, contents of effective ingredients and other ingredients present in the composition, type of formulation, age, weight, general health conditions, gender and diet of patients, administration time, administration routes, secretion ratio of the composition, treatment period, and the drug used in combination. In the method for preventing, treating or improving of the present invention, in case of an adult, the dose administered one or several times each day is preferably 0.001 g/kg to 1 g/kg.

The porous polymer microspheres according to the present invention can be manufactured using a pharmaceutically suitable and physiologically available adjuvant, in addition to the effective ingredients and examples of the adjuvant include vitamins, colorants, thickening agents, pectic acid, electrolytes, alginic acid, organic acids, carbonating agents, excipients, disintegrants, sweeteners, binders, coating agents, swelling agents, glydents, lubricants, or flavors.

The porous polymer microspheres further include one or more pharmaceutically available carriers, apart from the aforementioned active ingredient, and are thus preferably prepared into a pharmaceutical composition for administration.

Any formulation of the porous polymer microspheres may be used without particular limitation so long as it is a common formulation of pharmaceutical compositions, but is preferably an injectable liquid preparation (liquid solution). A pharmaceutically acceptable carrier regarding a composition prepared into the liquid solution is suitable for sterilization and is biocompatible, and examples thereof include saline, sterile water, Ringer's solution, buffered saline, albumin injection solutions, dextrose solutions, maltodextrin solutions, glycerol, ethanol and mixtures thereof. If necessary, the porous polymer microspheres may further an ordinary additive such as an antioxidant, buffer or fungistatic. Further, the porous polymer microspheres can be prepared into formulations for injection such as aqueous solutions, suspensions and emulsions, or pills, capsules, granules or tablets.

According to the present invention, the concentration of drug present in the porous polymer microspheres is preferably 1 ng/ml to 500 mg/ml and can be changed depending on type of contained drug. For example, when the application of the drug is for regenerating cartilage or bones, the concentration of drug is preferably 1 pg/ml to 3 mg/ml and when application of the drug is for inhibiting inflammation, the concentration of drug is preferably 1 μg/ml to 3 mg/ml. However, it is obvious that the concentration of drug can be easily changed depending on severity of diseased sites.

### MODE FOR INVENTION

Hereinafter, although preferred examples will be suggested for better understanding of the present invention, it is obvious to those skilled in the art that the following examples are provided only for illustration of the present invention, various alterations and modifications are possible within the range and technical concept of the present invention, and these alterations and modifications fall within the scope of the claims.

### Example 1. Production of Porous Polymer Microspheres (Containing Kartogenin)

140 mg of poly(lactide-co-glycolide) was dissolved in 7 g of dichloromethane to prepare a 2 wt % PLGA solution. 750 mg of gelatin was dissolved in 10 g of distilled water to prepare a 7.5 wt % aqueous gelatin solution. PLGA and gelatin were mixed in a weight ratio of 7:3 based on solids content, a solution of 10, 50 or 100 μM (each 0.05, 0.1 or 0.5 wt %) kartogenin in dimethylsulfoxide was added to the mixture, and the resulting mixture was homogenized in a homogenizer (Ultra-Turrax T-25 Basic, IKA) at 13,500 rpm for one minute to prepare an emulsion.

The emulsion as a discontinuous phase and a 0.5 wt % polyvinyl alcohol solution as a continuous phase were poured at a flow rate of 0.5 mL/min into a fluidic device to produce polymer microspheres. Distilled water was added to the produced microspheres and the mixture was stirred in 40° C. distilled water to release gelatin from the microspheres and thereby produce porous polymer microspheres containing kartogenin.

### Example 2. Production of Porous Polymer Microspheres 2 (Containing Ibuprofen)

2 wt % PLGA, a 3 wt % aqueous gelatin solution, and 1, 3 and 5 wt % ibuprofen solutions (in dichloromethane) were used, 0.5 wt % polyvinyl alcohol was further added as an emulsion stabilizer, and the mixture was homogenized in a homogenizer (Ultra-Turrax T-25 Basic, IKA) at 13,500 rpm for one minute to prepare an emulsion.

The emulsion as a discontinuous phase and 0.5 wt % polyvinyl alcohol as a continuous phase were poured at a flow rate of 0.5 mL/min into a fluidic device to produce polymer microspheres. Distilled water was added to the produced microspheres and the mixture was stirred in 45° C. distilled water to release gelatin from the microspheres and thereby produce porous polymer microspheres containing ibuprofen.

### Example 3. Production of Porous Polymer Microspheres 3 (Containing Curcumin)

Porous polymer microspheres were produced in the same manner as in Example 1, except that 0.5, 2 and 5 wt % curcumin was used in preparing the emulsion.

### Test Example 1. Morphological Analysis

### Test Example 1.1. Scanning Electron Microscopy

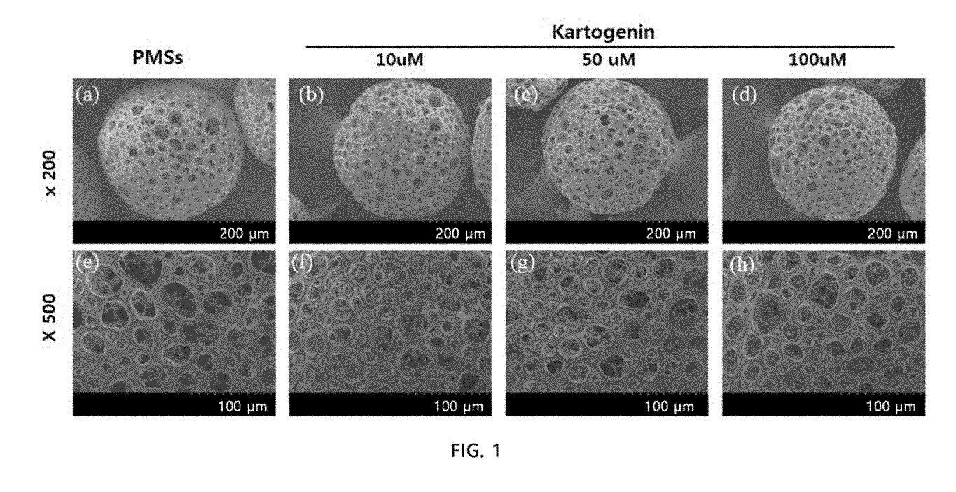

The morphologies of porous polymer microspheres produced by methods of Examples 1 to 3 were observed with a scanning electron microscope and results are shown in the following FIGS. 1 to 3.

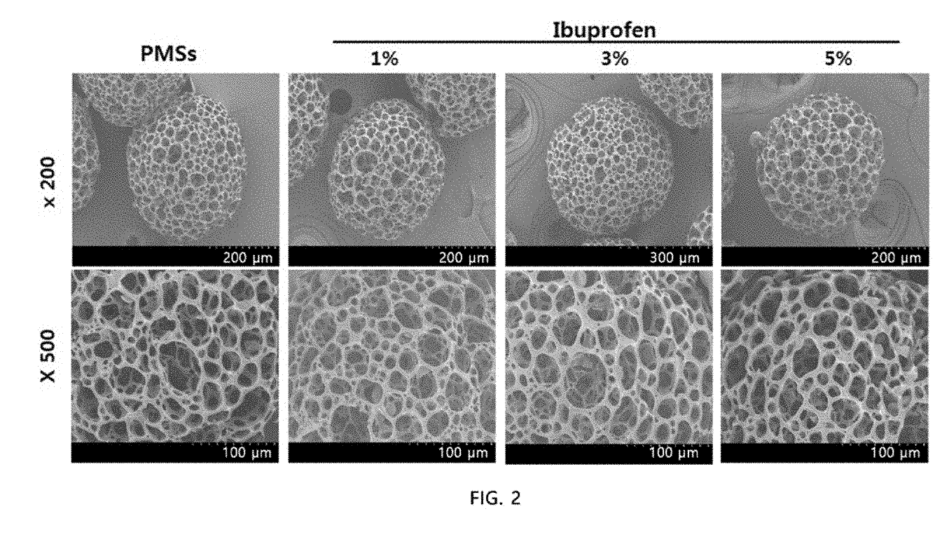

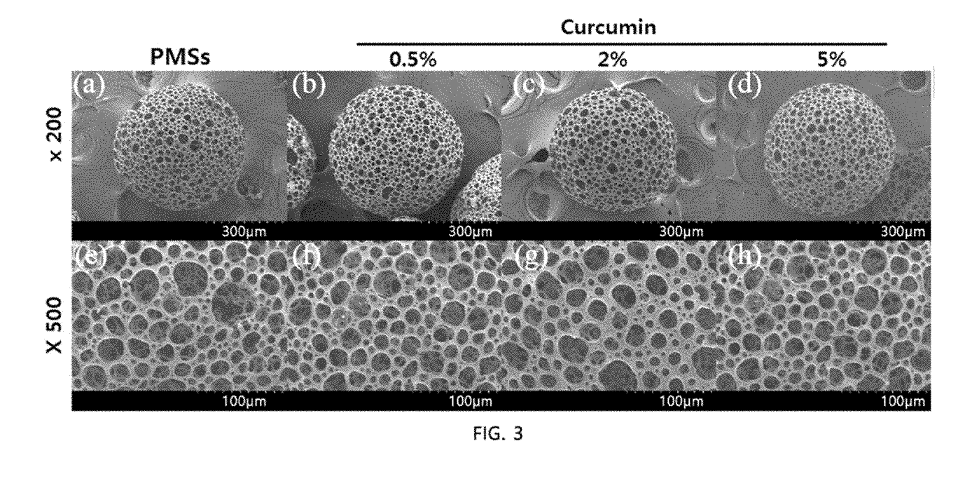

FIG. 1 is an image showing kartogenin-containing porous polymer microspheres (hereinafter referred to as “microspheres”) according to Example 1, FIG. 2 is an image showing ibuprofen-containing microspheres according to Example 2, and FIG. 3 is an image showing curcumin-containing microspheres according to Example 3. The surfaces and pore sizes of microspheres show similar behaviors at different concentrations, and show significant differences depending on drug concentration.

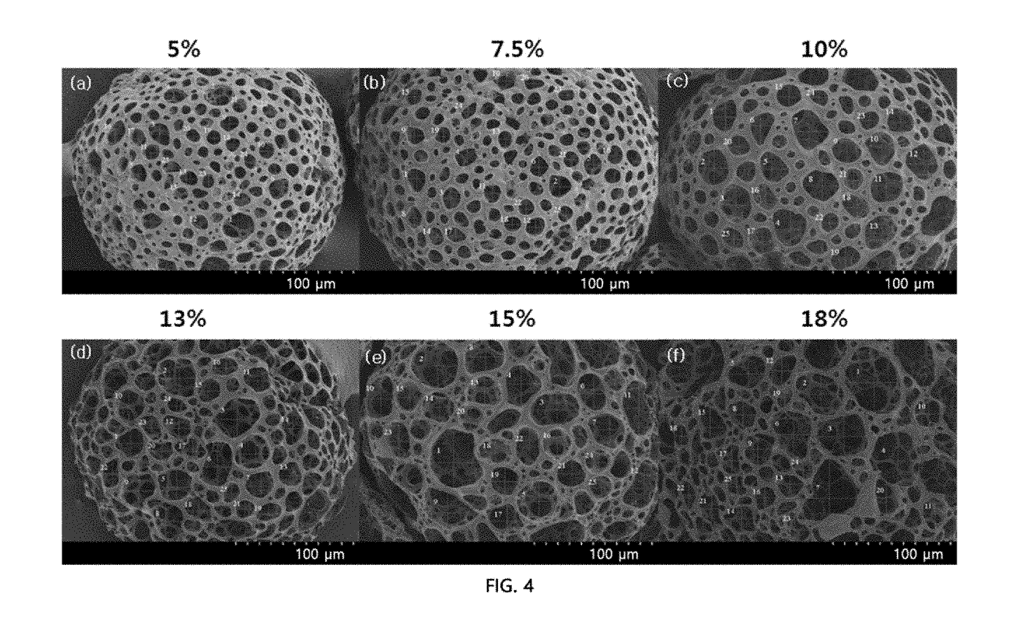

Meanwhile, in order to measure variations in pore size depending on concentration of drug, microspheres were produced while stepwise increasing the concentration of ibuprofen from 5% to 18%. The variations were measured with a scanning electron microscope, results are shown in the following FIG. 4 and Table 1, and variation in pores depending on curcumin concentration was observed and results are shown in the following Table 2.

TABLE 1

| | Ibuprofen concentration (wt %) | | | | | |
|---|---|---|---|---|---|---|
| Items | 5 | 7.5 | 10 | 13 | 15 | 18 |
| Pore width | 15.42 | 14.76 | 22.36 | 22.50 | 28.10 | 23.97 |
| (μm) | | | | | | |
| Pore height | 14.30 | 15.23 | 23.30 | 23.11 | 30.74 | 27.30 |
| (μm) | | | | | | |

TABLE 2

Curcumin concentration (wt %)

| Items | 0 | 0.5 | 2 | 5 |
|---|---|---|---|---|
| Pore width (μm) | 25.08 | 24.52 | 25.52 | 24.72 |
| Pore height (μm) | 24.6 | 24.64 | 25.44 | 24.76 |

As can be seen from Table 1, pore size increased depending on drug concentration, and there was no significant difference at a concentration of 7.5 wt % or less. Meanwhile, as can be seen from Tables 1 and 2, presence of the emulsion stabilizer and gelatin concentration affected the pore size of microspheres in the process of manufacturing microspheres.

### Test Example 1.2. Confocal Laser Scanning Microscopy

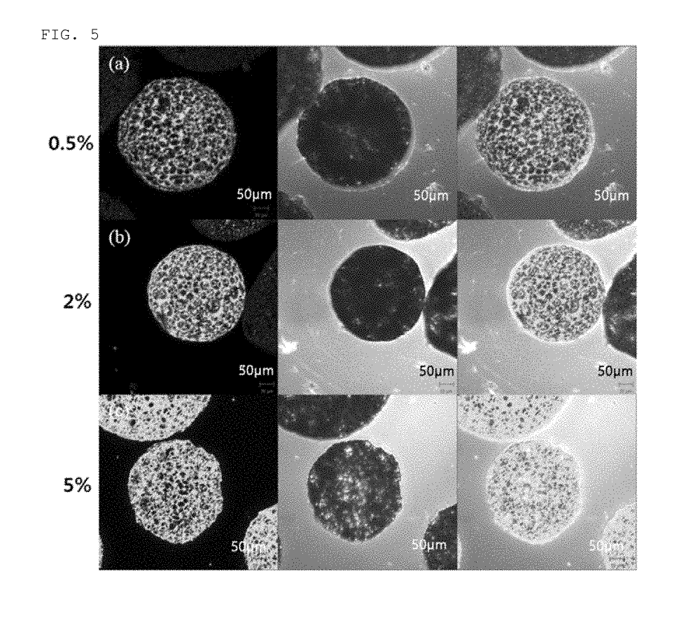

Microspheres of Example 3 were observed with a confocal laser scanning microscope and results are shown in FIG. 5. It could be seen that microspheres containing 5 wt % of curcumin had a denser internal structure than microspheres containing 0.5 wt % of curcumin.

### Test Example 1.3. Measurement of Particle Size and Porosity

The particle size and porosity of microspheres of Example 3 were observed and results are shown in the following Table 3. The particle size was 200 to 300 μm and the porosity was 81% on average.

TABLE 3

Curcumin concentration (wt %)

| Items | 0 | 0.5 | 2 | 5 |
|---|---|---|---|---|
| Particle size (μm) | 210.63 | 215.63 | 217.51 | 216.72 |
| Porosity (%) | 81.27 | 81.20 | 80.82 | 81.06 |

### Test Example 2. Drug Release Behaviors Test

### Test Example 2.1. Kartogenin Release Behaviors

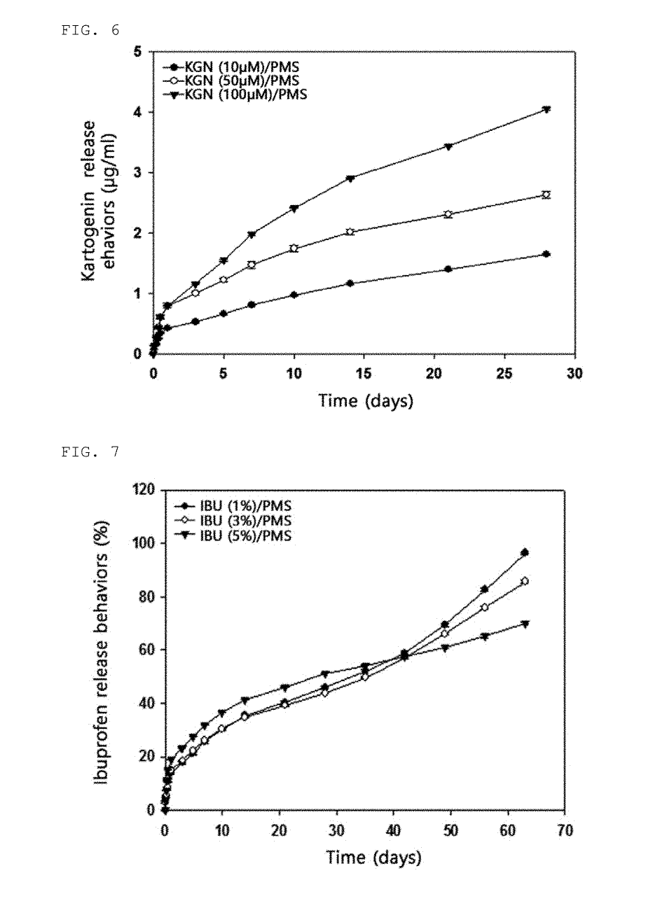

The drug release behaviors of the microspheres produced in Example 1 were observed. Specifically, while respective microspheres were stirred in 1 ml PBS buffer (pH 7.4) at 37° C. and 100 rpm, the amounts of released drug were measured at different times (1 hour, 3 hours, 6 hours, 10 hours, 1 day, 3 days, 5 days, 7 days, 10 days, 14 days, 21 days and 28 days). After measurement, the buffer was replaced with new buffer. The absorbance at 459 nm of released drug was measured to analyze the amount of released drug. Results are shown in the following FIG. 6.

As can be seen from FIG. 6, the amount of released drug increased dependent upon kartogenin concentration.

### Test Example 2.2. Ibuprofen Release Behaviors

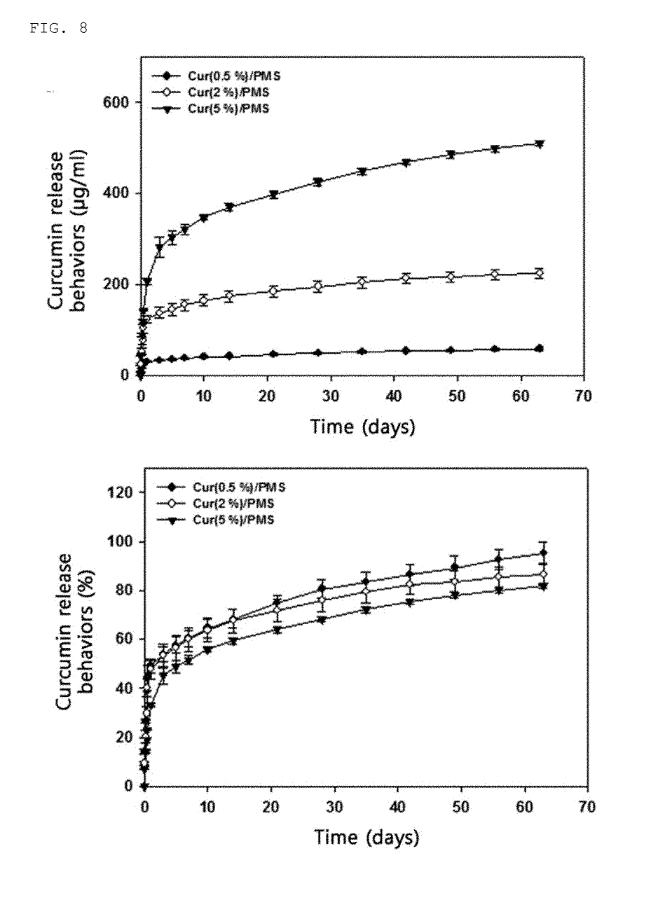

The drug release behaviors of the microspheres produced in Example 2 were observed in the same manner as in Example 1, except that drug release behaviors were observed up to 65 days. The absorbance at 359 nm of released drug was measured to analyze the amount of released drug. Results are shown in the following FIG. 7.

Ibuprofen was rapidly released from microspheres at an initial stage and its release rate became slower over time. Release rate tended to be not greatly affected by concentration until 43 days. Meanwhile, microspheres containing a low concentration of drug (1 wt % of ibuprofen) released most of the drug present therein in 65 days, whereas microspheres containing a high concentration of drug retained about 35% of the drug after 65 days.

### Test Example 2.3. Curcumin Release Behaviors

The drug release behaviors of the microspheres produced in Example 3 were observed. Specifically, while respective microspheres were stirred in 1 ml PBS buffer (pH 7.4) at 37° C. and 80 rpm, the amounts of released drugs were measured for a period of time (from 1 hour to 65 days). The absorbance at 420 nm of released drug was measured to analyze the amount of released drug. Results are shown in the following FIG. 8.

Like other drugs, the amount of released curcumin increased dependent upon curcumin concentration. However, in all cases, the release rate was high at an initial stage (1 day) and then became slower, although the release rate relative to the total weight was changed depending on concentration.

### Test Example 3. Identification of Inflammation Suppression Effect

Inflammation-induced cells were treated with microspheres according to Examples 1 to 3 and amounts of expressed inflammation markers were measured to identify inflammation suppression effect.

### Test Example 3.1 Kartogenin-Containing Microspheres

1×10⁵ stem cells were seeded on microspheres of Example 1, IL-1β was added to induce inflammation and the cells were cultured for 1 day and 3 days. After culture, the cells were washed with PBS buffer and then a Tris-EDTA solution, to isolate the cells from the microspheres. The isolated cells were centrifuged at 13,500 rpm for one minute, the triazol reagent and chloroform were injected into the cells and the reaction was carried out for 5 minutes. After reaction, the reaction solution was centrifuged at 13,500 rpm for 10 minutes and the supernatant was separated. The separated supernatant and isopropanol were mixed in a volume ratio of 1:1 and reaction was carried out for 5 minutes. After reaction, the reaction solution was centrifuged at 13,500 rpm for 10 minutes. The resulting white material was washed with ethanol and dried at room temperature, and RNA concentration was then measured at 260/280 nm with NanoDrop using diethyl pyrocarbonate (DEPC)-treated water. After isolation of RNA, the isolated RNA was analyzed by cDNA synthesis and real-time PCR using AccuPower® RT-PCR PreMix and the expression amounts of MMP3, MMP13, COX-2 and ADAMTS-5 as inflammation markers were measured.

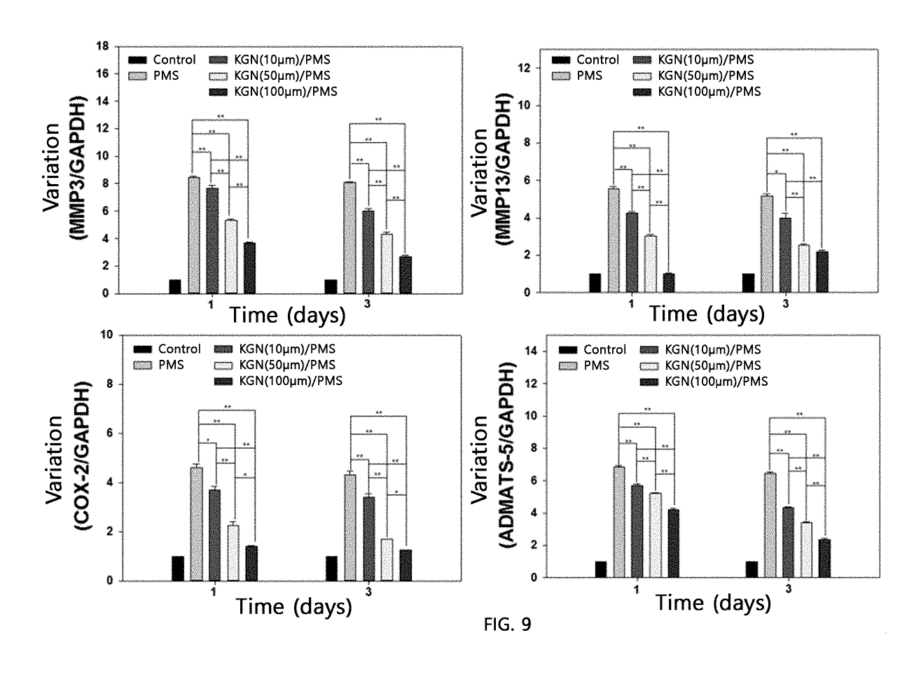

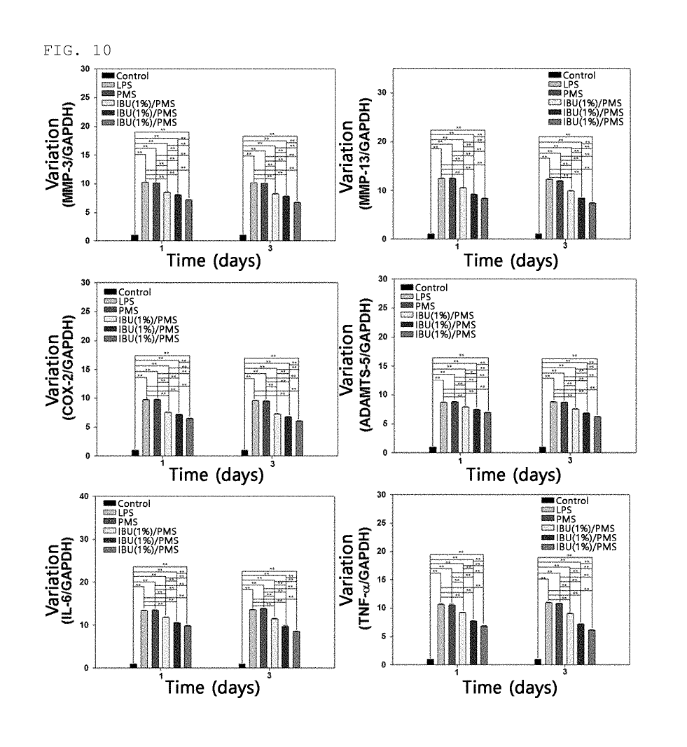

As can be seen from FIG. 9, the expression amounts of MMP3, MMP13, COX-2 and ADAMTS-5 decreased dependent upon kartogenin concentration.

### Test Example 3.2. Ibuprofen-Containing Microspheres

In the same manner as in Test Example 3.1, stem cells were seeded on microspheres of Example 2, IL-1β was added to induce inflammation, the cells were cultured and RNA was isolated from the microspheres. The isolated RNA was analyzed by cDNA synthesis and real-time PCR using AccuPower® RT-PCR PreMix, and the expression amounts of MMP3, MMP13, COX-2, ADAMTS-5, IL-6 and TNF-α as inflammation markers were measured.

As can be seen from FIG. 10, the expression amounts of MMP3, MMP13, COX-2, ADAMTS-5, IL-6 and TNF-α as inflammation markers decreased dependent upon ibuprofen concentration.

### Test Example 3.3. Curcumin-Containing Microspheres

In the same manner as in Test Example 3.1, 1×10⁵ tendon cells (tenocytes) were seeded on microspheres of Example 3, IL-1β was added to induce inflammation, the cells were cultured and RNA was isolated from the microspheres. The isolated RNA was analyzed by cDNA synthesis and real-time PCR using AccuPower® RT-PCR PreMix, and the expression amounts of MMP3, MMP13, COX-2 and ADAMTS-5 as inflammation markers were measured.

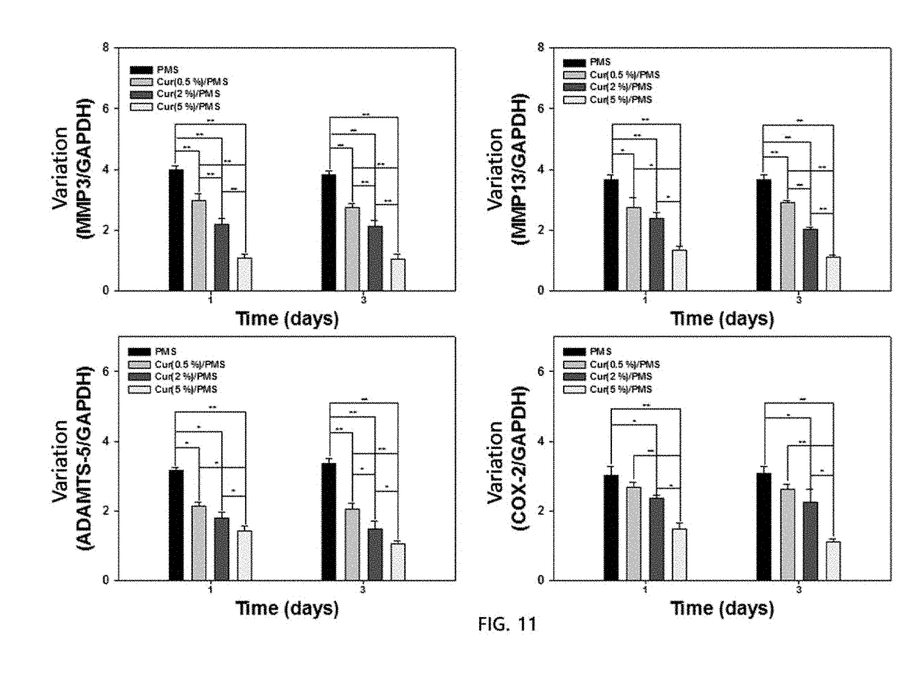

As can be seen from FIG. 11, the expression amounts of MMP3, MMP13, COX-2 and ADAMTS-5 serving as inflammation markers in tenocytes, which are one of soft tissues, decreased dependent upon the curcumin concentration.

### Test Example 4. Cartilage Differentiation Facilitation Test

1×10⁵ stem cells were seeded on microspheres of Example 1, cartilage differentiation buffer was added thereto and the cells were cultured for 21 days. After culture, the cells were washed with PBS buffer and then a Tris-EDTA solution, to isolate the cells from the microspheres. The isolated cells were centrifuged at 13,500 rpm for one minute, the triazol reagent and chloroform were injected into the cells and reaction was carried out for 5 minutes. After reaction, the reaction solution was centrifuged at 13,500 rpm for 10 minutes and the supernatant was separated therefrom. The separated supernatant and isopropanol were mixed in a volume ratio of 1:1 and reaction was carried out for 5 minutes. After reaction, the reaction solution was centrifuged at 13,500 rpm for 10 minutes. The resulting white material was washed with ethanol and dried at room temperature, and RNA concentration was measured at 260/280 nm with NanoDrop using diethyl pyrocarbonate (DEPC)-treated water. After isolation of RNA, the isolated RNA was analyzed by cDNA synthesis and real-time PCR using AccuPower® RT-PCR PreMix and the expression amounts of aggrecan, COL1A1, COL2A1 and COL10A1 as cartilage differentiation markers were measured.

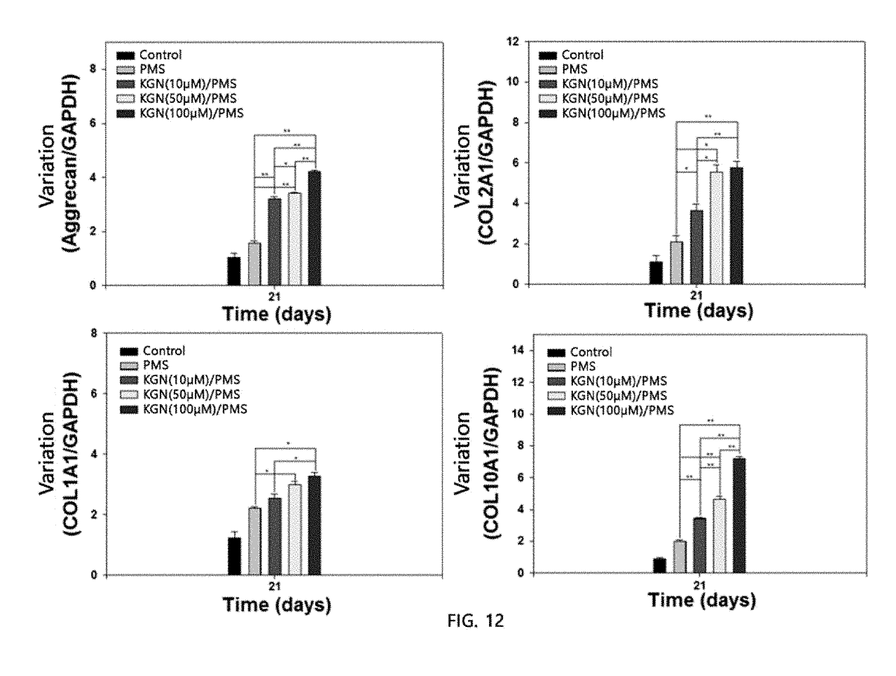

As can be seen from FIG. 12, the expression amounts of aggrecan, COL1A1, COL2A1 and COL10A1 as cartilage differentiation markers increased dependent upon kartogenin concentration.

### Test Example 5. In-Vivo Test

50 μl of monosodium iodoacetate (MIA) was injected and the effects of treatment of Example 2 on cartilage regeneration were identified using an animal model having damaged knee cartilage.

190-210 g weighed-SPF (specific pathogen free) 6 week-aged rats (n=30) were purchased and were allowed to be freely feed on solid feeds and tap water at a relative humidity of 22±2° C. and 55±10% on a bright-dark cycle of 12 hours over an adaptation period of one week. The rats were arbitrarily divided into six groups. One week after knee damage, the rats were treated with the drug for 8 weeks.

Normal group: Non-treatment group

Control group: Group to which distilled water is administered after knee damage,

Drug-administration group: Group to which 200 μl of microspheres containing ibuprofen (0, 1, 3 and 5% of ibuprofen) are administered after knee damage

### Test Example 5.1. X-Ray and Micro CT Imaging

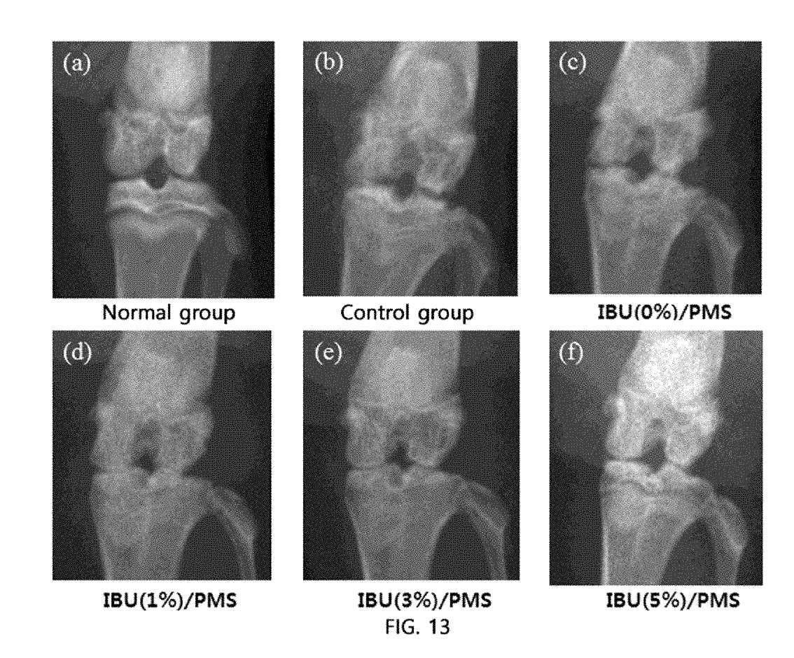

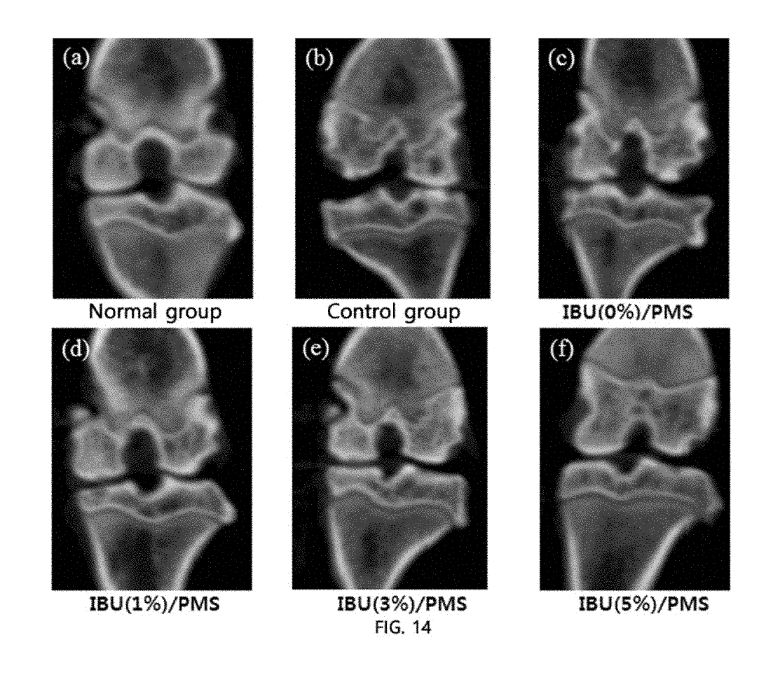

Knees of subject animals were imaged by X-ray and micro CT and the results are shown in FIGS. 13 and 14. Also, excised knees were imaged and results are shown in FIG. 14.

a: Normal group, b: Control group, c-f: Drug-administration group (0, 1, 3, 5 wt %)

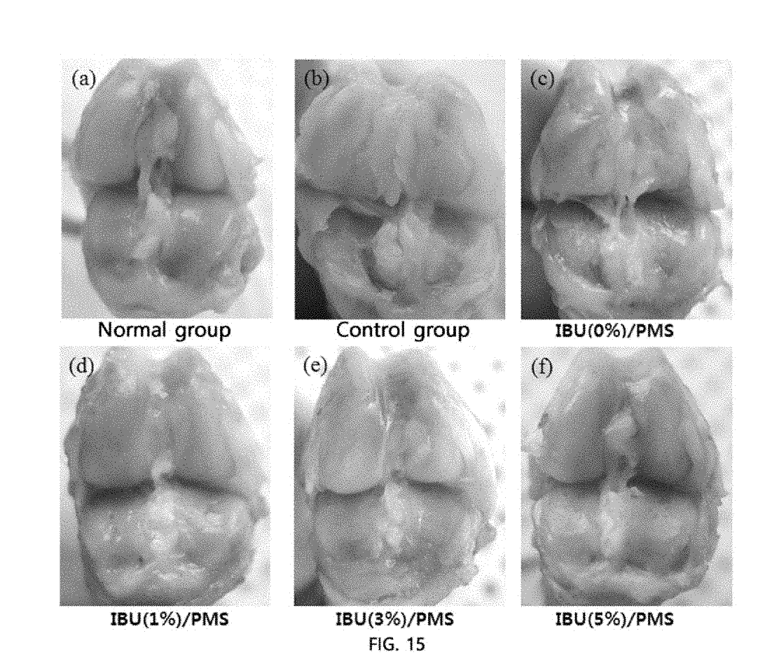

As can be seen from FIGS. 13 to 15, Control group (b) and Test group (c) of microspheres containing no ibuprofen exhibited serious damage to cartilage, but Drug-administration group (d-f) of microspheres containing ibuprofen involved cartilage production dependent upon ibuprofen concentration.

### Test Example 5.2. Histological Examination

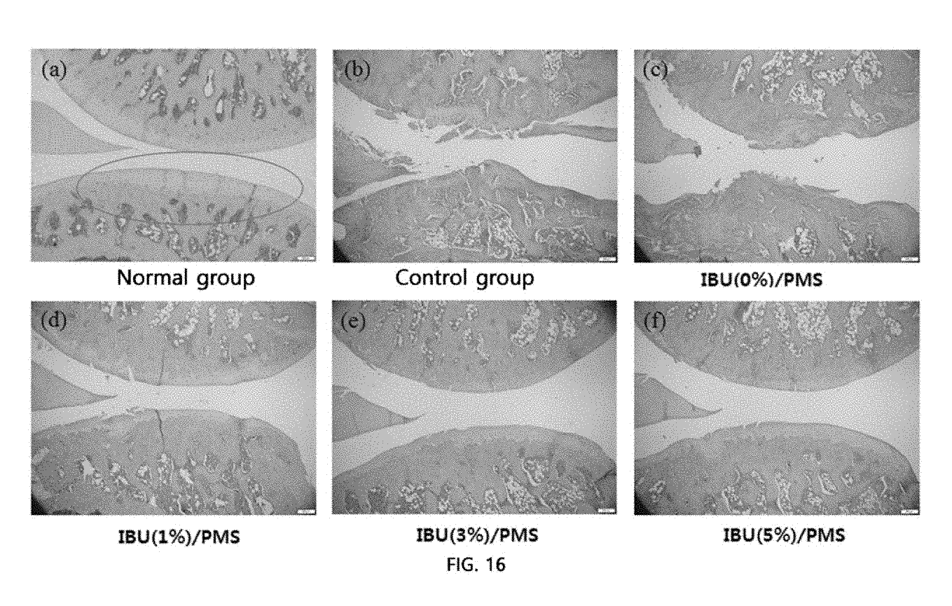

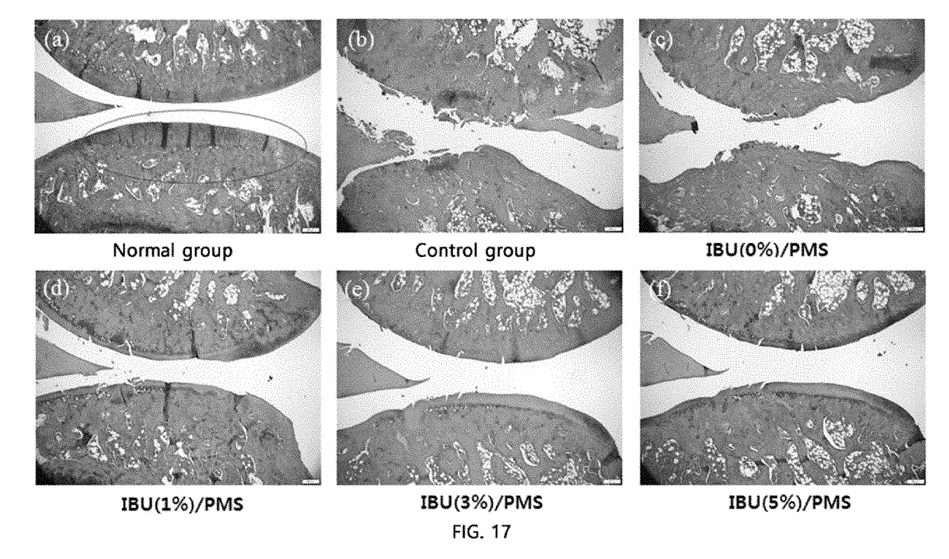

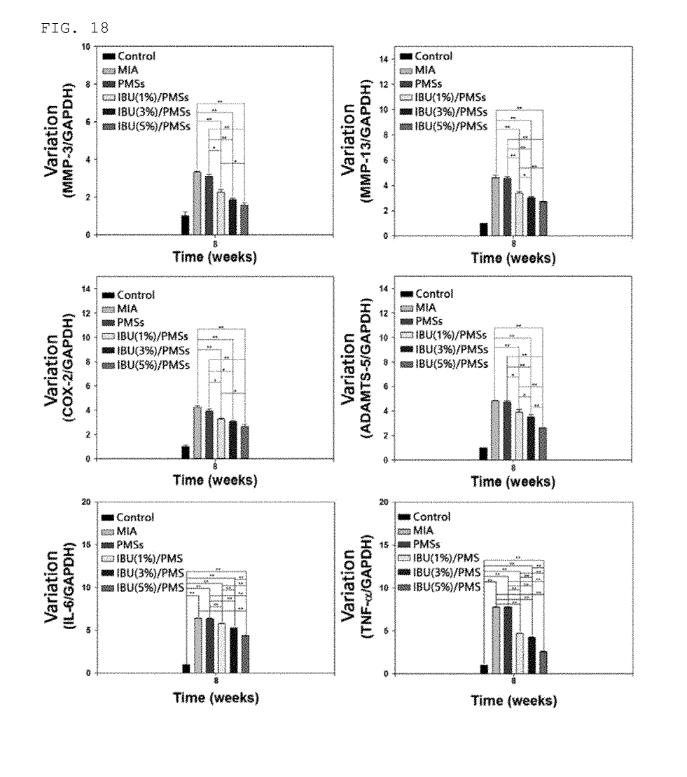

The animals were sacrificed and the knees were excised therefrom and immobilized with 10% formalin for 2 weeks to produce paraffin blocks. Fragments (5 μm) were prepared and stained with hematoxylin & eosin (H&E) and safranin O, and results are shown in FIG. 16 and FIG. 17, respectively.

a: Normal group, b: Control group, c-f: Drug-administration group (0, 1, 3, 5 wt %)

As can be seen from FIGS. 16 and 17, Control group (b) and Test group (c) of microspheres containing no ibuprofen had a mountain-like shape, since cartilage was removed by MIA treatment. On the other hand, Drug-administration group (d-f) had production of cartilage dependent upon ibuprofen concentration.

Although damage to cartilage was serious, Group to which microspheres containing ibuprofen were administered (d-f) had production of cartilage dependent upon ibuprofen concentration.

### Test Example 5.3. Test on Concentrations of Inflammation Markers Expressed in Blood

The expression amounts of MMP3, MMP13, COX-2, ADAMTS-5, IL-6 and TNF-α as inflammatory cytokines were measured using blood acquired from the ventral aorta of test animals and results are shown in FIG. 18.

Similar to in vitro, MMP3, MMP13, COX-2, ADAMTS-5, IL-6 and TNF-α as inflammation markers decreased dependent upon ibuprofen concentration.

1. Porous polymer microspheres for preventing or treating soft tissue diseases comprising:
a biodegradable polymer scaffold having a three-dimensional network structure in which pores having a size of 5 to 100 μm are connected to one another; and
a drug for treating soft tissue diseases incorporated in the network structure of the biodegradable polymer scaffold.

2. The porous polymer microspheres according to claim 1, wherein the drug is incorporated in an amount of 2 to 30 parts by weight with respect to 10 parts by weight of the biocompatible polymer.

3. The porous polymer microspheres according to claim 1, wherein the porous polymer microspheres have a porosity of 10 to 90% and a particle size of 200 to 1,000 μm.

4. The porous polymer microspheres according to claim 1, wherein the porous polymer microspheres have a porosity of 60 to 90%, a pore size of 10 to 40 μm and a particle size of 200 to 500 μm.

5. The porous polymer microspheres according to claim 1, wherein the drug for treating soft tissue diseases is selected from transforming growth factor (TGF), platelet derived growth factor (PDGF), basic fibroblast growth factor (bFGF), vascular endothelial growth factor (VEGF), insulin like growth factor (IGF), bone morphogenetic protein-7 (BMP-7), anti-inflammatory peptide, aspirin, mefenamic acid, diclofenac sodium, indomethacin, naproxen, acetaminophen, ketoprofen, loxoprofen, piroxicam, ibuprofen, simvastatin, atorvastatin, fluvastatin, lovastatin, copper-peptide, prostaglandin, tramadol, celecoxib, glucosamine, chondroitin, diacerein, methotrexate, cyclosporine, an Janus Kinase 3 inhibitor (JAK-inhibitor), rituximab, tocilizumab, salazosulfapyridine (SASP), bucillamine, leflunomide, infliximab, etanercept, adalimumab, prednisolone, antiflammin 2, curcumin, sulfasalazine, lactoferrin, kartogenin, ibuprofen or a mixture thereof.

6. The porous polymer microspheres according to claim 1, wherein the biocompatible polymer is selected from the group consisting of: any one selected from polyglycolic acid (PGA), polylactic acid (PLA), poly(lactide-co-glycolide) (PLGA), polycaprolactone (PCL), polyamino acid, polylactide, polyphosphazine, polyiminocarbonate, polyphosphoester, polyanhydride, polyorthoester, polyhydroxyvalerate, polyhydroxybutyrate, hyaluronic acid, cellulose, heparin, collagen, alginate and chitosan; polymers blended with two or more thereof; and copolymers of two or more thereof.

7. The porous polymer microspheres according to claim 1, wherein the soft tissue disease is selected from Achilles tendinitis, rotator cuff tendinitis, patellar tendinitis, sprains, ligamentitis, degenerative osteoarthritis and inflammatory arthritis.

8. The porous polymer microspheres according to claim 1, wherein the porous polymer microspheres release the drug when injected in vivo.

9. The porous polymer microspheres according to claim 5, wherein the drug for treating soft tissue diseases is kartogenin, ibuprofen, curcumin or a mixture thereof.

10. A method for manufacturing porous polymer microspheres for preventing or treating soft tissue diseases comprising:
1) dissolving a biocompatible polymer and a drug for treating soft tissue diseases in an organic solvent to prepare a polymer solution;
2) homogenizing the polymer solution and an aqueous gelatin solution to prepare an emulsion;
3) pouring the emulsion as a discontinuous phase and a polyvinyl alcohol solution as a continuous phase into a fluidic device to produce polymer microspheres including the biocompatible polymer, the drug and gelatin; and
4) stirring the polymer microspheres in 38-50° C. water to release the gelatin from the polymer microspheres.

11. The method according to claim 10, wherein the drug is incorporated in an amount of 2 to 30 parts by weight with respect to 10 parts by weight of the biocompatible polymer.

12. The method according to claim 10, wherein in homogenization, 3 to 15 parts by weight of the gelatin is added with respect to 10 parts by weight of the biocompatible polymer.

13. The method according to claim 10, wherein the biocompatible polymer is selected from the group consisting of: any one selected from polyglycolic acid (PGA), polylactic acid (PLA), poly(lactide-co-glycolide) (PLGA), polycaprolactone (PCL), polyamino acid, polylactide, polyphosphazine, polyiminocarbonate, polyphosphoester, polyanhydride, polyorthoester, polyhydroxyvalerate, polyhydroxybutyrate, hyaluronic acid, cellulose, heparin, collagen, alginate and chitosan; polymers blended with two or more thereof; and copolymers of two or more thereof.

14. The method according to claim 10, wherein, in step 2), 0.5 to 3 parts by weight of an emulsion stabilizer is further added.

15. The method according to claim 10, wherein the porous polymer microspheres have a porosity of 10 to 90%, a pore size of 5 to 100 μm and a particle size of 200 to 1,000 μm.

16. The method according to claim 10, wherein the porous polymer microspheres have a porosity of 60 to 90%, a pore size of 10 to 40 μm and a particle size of 100 to 400 μm.

17. The method according to claim 10, wherein the soft tissue disease is selected from Achilles tendinitis, rotator cuff tendinitis, patellar tendinitis, sprains, ligamentitis, degenerative osteoarthritis and inflammatory arthritis.

18. The method according to claim 10, wherein, in step 2), homogenization is carried out using a homogenizer at 8,000 to 20,000 rpm for 30 seconds to 10 minutes.

19. The method according to claim 10, wherein a release amount and rate of the drug are regulated by controlling a content of the gelatin and/or the drug with respect to a content of the biocompatible polymer.

20. The method according to claim 10, wherein the aqueous gelatin solution is a 1 to 10 wt % aqueous gelatin solution.

21. The method according to claim 10, wherein the discontinuous phase and the continuous phase are each poured at a flow rate of 0.2 to 1 mL/min.

22. The method according to claim 10, wherein the organic solvent comprises one or more selected from dimethylsulfoxide, dichloromethane, tetrahydrofuran and N,N-dimethylformamide.

23. The method according to claim 10, wherein the drug for treating soft tissue diseases is selected from transforming growth factor (TGF), platelet derived growth factor (PDGF), basic fibroblast growth factor (bFGF), vascular endothelial growth factor (VEGF), insulin like growth factor (IGF), bone morphogenetic protein-7 (BMP-7), anti-inflammatory peptide, aspirin, mefenamic acid, diclofenac sodium, indomethacin, naproxen, acetaminophen, ketoprofen, loxoprofen, piroxicam, ibuprofen, simvastatin, atorvastatin, fluvastatin, lovastatin, copper-peptide, prostaglandin, tramadol, celecoxib, glucosamine, chondroitin, diacerein, methotrexate, cyclosporine, a Janus Kinase 3 inhibitor (JAK-3 inhibitor), rituximab, tocilizumab, salazosulfapyridine (SASP), bucillamine, leflunomide, infliximab, etanercept, adalimumab, prednisolone, antiflammin 2, curcumin, sulfasalazine, lactoferrin and kartogenin, ibuprofen or a mixture thereof.
